(19) United States
(12) Patent Application Publication
(10) Pub. No.: US 2025/0212887 A1
(43) Pub. Date: Jul. 3, 2025

(54) RECOMBINANT ENTOMOPATHOGENIC BACTERIA PREPARED USING PROMOTER REPLACEMENT TECHNIQUE, PREPARATION METHOD, AND USES THEREOF

(71) Applicant: Andong National University Industry-Academic Cooperation Foundation, Andong-si (KR)

(72) Inventors: Yong Gyun KIM, Andong-si (KR); Ga Hyeon JIN, Andong-si (KR)

(73) Assignee: Andong National University Industry-Academic Cooperation Foundation, Andong-si (KR)

(21) Appl. No.: 18/974,557

(22) Filed: Dec. 9, 2024

(30) Foreign Application Priority Data
Dec. 28, 2023 (KR) 10-2023-0194288

(51) Int. Cl. A01N 63/20; A01N 37/46; A01P 7/04; C12N 1/20; C12N 9/00; C12N 15/74; C12N 15/90

(52) U.S. Cl. CPC A01N 63/20; A01N 37/46; A01P 7/04; C12N 1/20; C12N 9/93; C12N 15/74; C12N 15/902; C12Y 600/00

(57) ABSTRACT

Proposed are recombinant entomopathogenic bacteria transformed using promoter replacement technique, a preparation method, and uses thereof for producing pesticides and controlling pests. Provided are recombinant entomopathogenic bacteria in which the promoter of the GXP synthase gene (gxpS gene) is replaced in the entomopathogenic bacteria with an arabinose inducible promoter using a promoter replacement technique. The recombinant entomopathogenic bacteria prepared in the present disclosure can produce large quantities of GXPs, which acts to suppress immunity of pests, by controlling the expression of the GXP synthetase gene (gxpS gene) through an addition of an inducer. The recombinant entomopathogenic bacteria prepared in the present disclosure can also effectively control pests through a response that suppresses the immunity of pests.

### CROSS REFERENCE TO RELATED APPLICATION

The present application claims priority to Korean Patent Application No. 10-2023-0194288, filed Dec. 28, 2023, the entire contents of which is incorporated herein for all purposes by this reference.

### REFERENCE TO AN ELECTRONIC SEQUENCE LISTING

The contents of the electronic sequence listing (PANY-129-SequenceListing.xml; Size: 3,227 bytes; and Date of Creation: Dec. 28, 2023) is herein incorporated by reference in its entirety.

### STATEMENT REGARDING GOVERNMENT SPONSORED RESEARCH

This invention was supported by the Korea Institute of Planning and Evaluation for Technology in Food, Agriculture and Forestry funded by the Ministry of Agriculture, Food and Rural Affairs. [Research Project name: “Development of Industrialized Technology to Control Plant Virus, Disease and Insect Pests”; Research Subject name: “Control techniques against insect pests infesting green onion using insect pathogenic microbes”; Project Serial Number: 1545026738; Research Subject Number: 321100033SB010]

### BACKGROUND OF THE DISCLOSURE

### 1. Field of the Disclosure

The present disclosure relates to recombinant entomopathogenic bacteria transformed using a promoter replacement technique, a preparation method, and uses thereof for producing pesticides and controlling pests.

### 2. Description of the Related Art

The entomopathogenic bacterial genera Photorhabdus and Xenorhabdus coexist with the entomopathogenic nematodes Heterorhabditis and Steinernema, respectively (Boemare et al., 2002). Bacteria of the genera Photorhabdus and Xenorhabdus are considered to have evolved independently from an evolutionary perspective, but the bacteria are presumed to share a common lifestyle due to convergent evolution (Chaston et al., 2011). Infective juveniles (IJ) of entomopathogenic nematodes enter a host insect through natural openings such as the mouth, anus, and stomata and ultimately infect the insect's blood cavity. The entomopathogenic nematodes then release symbiotic entomopathogenic bacteria from the intestine of the insect into the insect's hemolymph. The released entomopathogenic bacteria produce secondary metabolites that suppress the insect's immune response and protect the nematodes and the bacteria themselves in a mutualistic form (Nebacher et al., 2020). The entomopathogenic bacteria, which grow when the insect's immune response is suppressed, cause sepsis and kill the insect. These entomopathogenic nematodes, which are third-instar larvae developing from these killed insects, recombine with symbiotic entomopathogenic bacteria and transform into infective juveniles (IJ) (Stock, 2019). Therefore, secondary metabolites synthesized by entomopathogenic bacteria play an important role in the mutual symbiosis between the entomopathogenic nematodes and the entomopathogenic bacteria.

Secondary metabolites produced by the entomopathogenic bacteria include amino acid derivatives and peptides synthesized using polyketide synthetase (PKS), non-ribosomal peptide synthetase (NRPS), or amino acid derivatives synthesized using other enzymes, peptides, polyketides, and hybrid natural products (Cimen et al., 2022). The genome of entomopathogenic bacteria encodes many genes for producing these secondary metabolites (Shi et al., 2022).

GameXPeptides (GXPs), which are a type of secondary metabolites of these entomopathogenic bacteria, are a cyclic pentapeptide. Four GXPs, GXP-A to GXP-D, were first discovered (Bode et al., 2012). Later, four additional GXPs, GXP-E to GXP-H, were identified in Photorhabdus luminescens TTO1 under different bacterial culture conditions (Nollmann et al., 2015). GXPs are secondary metabolites commonly produced in Xenorhabdus and Photorhabdus (Tobias et al., 2017). The GXPs are assumed to play a unique role in these two bacterial genera, playing an important and common role in the pathogenicity cycle of nematode-bacterial mutualism (Shi and Bode, 2018). Shi et al. (2022) confirmed that GXP-A suppressed cellular immune responses, as measured by hemocyte-spreading action and vesicle formation. On the basis of this, Shi et al. proposed that GXP-A had an inhibitory activity on insect immunity. This immunosuppressive activity of GXP-A was confirmed to increase the insecticidal power of Bacillus thuringiensis against the lepidopteran insect Spodoptera exigua. This is because insect immunity is one of the infection barriers to bacterial pathogenicity (Hrithik et al., 2022). The GXPs are produced by catalytic activity of a GXP synthase, which is an NRPS produced by a specific synthase gene (gxpS gene) (Nollmann et al., 2015). However, whether expression of the gxpS gene is functionally related to bacterial toxicity in the insect has not been studied.

### RELATED ART DOCUMENTS

### Patent Documents

(Patent document 0001) Korean Patent Application Publication No. 10-2014-0044435

(Patent document 0002) Korean Patent Application Publication No. 10-2012-0136523

(Patent document 0003) Korean Patent Application Publication No. 10-2012-0091949

(Patent document 0004) Korean Patent Application Publication No. 10-2004-0104872

(Patent document 0005) Korean Patent No. 10-1362211

### SUMMARY OF THE DISCLOSURE

The present disclosure is to provide a recombinant entomopathogenic bacteria in which a promoter of a GXP synthase gene (gxpS gene) in an entomopathogenic bacteria is replaced with an arabinose inducible promoter and to provide a method of preparing the same.

In addition, the present disclosure is to provide uses for the recombinant entomopathogenic bacteria for controlling pests or producing GXPs by using the recombinant entomopathogenic bacteria.

In one aspect of the present disclosure, provided is recombinant entomopathogenic bacteria in which a promoter of a GXP synthase gene (gxpS gene) is replaced in entomopathogenic bacteria with an arabinose inducible promoter using a promoter replacement technique.

In a preferred embodiment, the entomopathogenic bacteria may be Photorhabdus temperata temperata. In another embodiment, the recombinant entomopathogenic bacteria may be Photorhabdus temperata temperata deposited as KACC 92578P.

In the recombinant entomopathogenic bacteria, the arabinose inducible promoter may preferably be araBAD.

The recombinant entomopathogenic bacteria may preferably mass-produce GXPs under control of the arabinose.

In another aspect of the present disclosure, provided is a method of preparing recombinant entomopathogenic bacteria, the method including:
amplifying an initial ORF region of a GXP synthase gene (gxpS gene) of entomopathogenic bacteria;
obtaining a recombinant vector by cloning the amplified initial ORF region into a vector under an arabinose inducible promoter;
obtaining a transformed E. coli by bringing the recombinant vector to be inserted into E. coli;
transferring the recombinant vector to the entomopathogenic bacteria by co-culturing the entomopathogenic bacteria and the transformed E. coli and facilitating bacterial conjugation thereof; and
obtaining recombinant entomopathogenic bacteria, which are homologous recombinants, by bringing the recombinant vector to be inserted into a genome of the entomopathogenic bacteria through homologous recombination.

In a further preferred embodiment of the preparation method, the entomopathogenic bacteria may be Photorhabdus temperata temperata. In a yet further embodiment, the recombinant entomopathogenic bacteria may be Photorhabdus temperata temperata deposited as KACC 92578P.

In the preparation method, obtaining a single colony by culturing the recombinant entomopathogenic bacteria may be further included.

In the preparation method, the amplifying of the ORF region may preferably be performed by amplifying the initial ORF region (600 bp) located at the 5′ end of the gxpS gene encoding the GXP synthetase of the entomopathogenic bacteria using a forward primer of SEQ ID NO: 1 and a reverse primer of SEQ ID NO: 2.

In the preparation method, the arabinose inducible promoter may preferably be araBAD.

In the preparation method, the vector may preferably be a pCEP plasmid.

In the preparation method, the E. coli may preferably be E. coli S17.

In the preparation method, homologous recombinants may preferably be formed at overlapping sequences involved in replacing promoters by bringing the recombinant vector to be inserted into a genome of the entomopathogenic bacteria through homologous recombination.

In the preparation method, the single colony may preferably be obtained by spreading recombinant Photorhabdus temperata temperata on LB agar medium containing kanamycin and culturing the recombinant Photorhabdus temperata temperata in a temperature range of 28° C. to 32° C. for 1 to 3 days.

In the preparation method, the recombinant Photorhabdus temperata temperata may preferably be confirmed to have sequencing primers by a PCR analysis using mutation-specific primers.

In a further aspect of the present disclosure, a method of producing GXPs using the recombinant entomopathogenic bacteria may be provided.

In the preparation method, the GXP production may preferably be controlled by arabinose.

In a yet further aspect of the present disclosure, a pest control method is provided, the method including treating pests with the recombinant entomopathogenic bacteria or GXP-A.

In a yet further preferred embodiment of the pest control method, the GXP-A may be obtained through the production method while insecticidal functions of the GXP-A have already been confirmed.

In a still yet further preferred embodiment of the pest control method, the recombinant entomopathogenic bacteria and Bacillus thuringiensis may be co-treated.

In a still yet further preferred embodiment of the pest control method, the pests may include any one selected from the group consisting of Spodoptera exigua, Plutella xylostella, Frankliniella occidentalis, and Aedes albopictus.

The preset disclosure prepares recombinant entomopathogenic bacteria capable of controlling GXP production by replacing a promoter of a GXP synthase gene (gxpS gene) with an arabinose inducible promoter using a promoter replacement technique.

The prepared recombinant entomopathogenic bacteria regulates expression of the GXP synthase gene (gxpS gene) by an inducer, arabinose, thereby the prepared recombinant entomopathogenic bacteria can produce large quantities of GXPs, which act to suppress immunity of pests.

When using the recombinant entomopathogenic bacteria which produce large quantities of the GXPs, which suppresses the immunity of pests, pests can be effectively controlled through a response, which suppresses the immunity of pests.

### BRIEF DESCRIPTION OF THE DRAWINGS

FIG. 1 shows a graph confirming a relationship between Photorhabdus temperata temperata growth (measured by OD600) and a relative expression level of a gxpS gene depending on bacterial culture time when cultured in TSB medium;

FIG. 2 shows a graph confirming the relationship between the Photorhabdus temperata temperata growth (measured in CFU) and the relative expression level of the gxpS gene in a hemocoel of Spodopte raexigua;

FIG. 3 shows a chromatogram of four GXPs (GXP-A, GXP-B, GXP-C, and GXP-D) confirmed by LC-MS/MS spectra using chemical structures of the GXPs;

FIG. 4 shows a graph showing quantification of the four GXPs (GXP-A, GXP-B, GXP-C, and GXP-D) contained in Photorhabdus temperata temperata culture medium cultured for 72 hours in TSB medium;

FIG. 5 shows a diagram showing a process of generating a mutant of a Photorhabdus temperata temperata gxpS gene;

FIG. 6 shows results of PCR analysis of the mutant of the Photorhabdus temperata temperata gxpS gene using sequencing primers;

FIG. 7 shows relative expression levels of the gxpS gene in the wild Photorhabdus temperata temperata and recombinant Photorhabdus temperata temperata, in which “−ARA” indicates no addition of ARA, and “+ARA” indicates addition of ARA;

FIG. 8 shows comparisons of amounts of four GXPs among wild and recombinant temperata temperata culture media;

FIGS. 9 and 10 show results of a hemocyte-spreading analysis to observe F-actin (Factin) growth in response to Photorhabdus temperata temperata extracts;

FIG. 11 shows results of confirming vesicle formation responses in response to infection with Photorhabdus temperata temperata;

FIG. 12 shows results of analyzing phenoloxidase (PO) activity in response to infection with Photorhabdus temperata temperata;

FIG. 13 shows results of comparing insecticidal activity against Spodoptera exigua in the following cases: when wild Photorhabdus temperata temperata was treated, when recombinant Photorhabdus temperata temperata was treated, and when recombinant Photorhabdus temperata temperata and GXP-A (10 μg/larva) were co-treated; and

FIG. 14 shows results of confirming mortality rates of Spodoptera exigua depending on different doses of GXP-A when the recombinant Photorhabdus temperata temperata (1.5×10² CFU/larva) and GXP A were co-treated.

### DESCRIPTION OF THE PREFERRED EMBODIMENTS

One aspect of the present disclosure provides recombinant entomopathogenic bacteria and a method of preparing the same. The recombinant entomopathogenic bacteria are transformed to control GXP production using a promoter replacement technique. The present disclosure will be described in detail below through preferred embodiments.

In a preferred embodiment of the present disclosure, obtained are recombinant Photorhabdus temperata temperata capable of producing large quantities of GXPs by confirming GXP production of the Photorhabdus temperata temperata, entomopathogenic bacteria and replacing a promoter of a GXP synthase gene (gxpS gene) with an arabinose inducible promoter in Photorhabdus temperata temperata. In another preferred embodiment of the present disclosure, Photorhabdus temperata temperata are used as the entomopathogenic bacteria, but the entomopathogenic bacteria capable of being used for the purposes of the present disclosure are not limited to Photorhabdus temperata temperata.

In one example, a Photorhabdus temperata temperata ANU101 strain (accession number KACC91042) isolated from Heterorhabditis megidis has been used as the entomopathogenic bacteria.

Photorhabdus temperata temperata is preferably cultured in a shaking incubator using trypsin soy bean (TSB) medium at a temperature of 28±2° C. and a shaking speed of 160 to 200 rpm for 70 to 74 hours. It is more preferable for Photorhabdus temperata temperata to be cultured for 72 hours at a temperature of 28° C. and a shaking speed of 180 rpm.

Then, using the promoter replacement technique, the promoter of the GXP synthase gene (gxpS gene) in Photorhabdus temperata temperata is replaced with the arabinose inducible promoter, thereby preparing recombinant Photorhabdus temperata temperata.

In Photorhabdus temperata temperata, the GXP synthase gene (gxpS gene) produces four types of GPX: GPX-A, GPX-B, GPX-C, and GPX-D. Out of the four GPXs, GPX-A is most produced.

Thereafter, first, an initial open reading frame region (ORF region) (600 bp) of the GXP synthase gene (gxpS gene) in Photorhabdus temperata temperata is amplified.

The initial ORF region (600 bp) located at the 5′ side of the GXP synthase gene (gxpS gene) in Photorhabdus temperata temperata is amplified by a PCR reaction.

At this time, as primers, it is preferable to use a forward primer of SEQ ID NO: 1 and a reverse primer of SEQ ID NO: 2 shown on a sequence list.

The forward has a sequence primer of SEQ ID NO: 1 (CATATGATGAAAGACAGTATTACCAG) and includes an Nde I restriction enzyme site (underlined).

The reverse primer of SEQ ID NO: 2 has a sequence (CTGCAGCATGATATACGCCGGCCCCGG) and includes a PstI restriction site (underlined).

The amplified initial ORF region (600 bp) is cloned into a vector under the arabinose inducible promoter, thereby obtaining a recombinant vector.

The inducible promoter inducible by arabinose is preferably araBAD, an arabinose inducible promoter.

It is preferable to use a pCEP plasmid as a vector. The recombinant vector is inserted into E. coli, thereby obtaining transformed E. coli. At this time, E. coli S17 may be used for the E. coli.

The Photorhabdus temperata temperata and the transformed E. coli are co-cultured and bacterially conjugated to transfer the recombinant vector to the Photorhabdus temperata temperata.

The recombinant vector transferred to the Photorhabdus temperata temperata through bacterial conjugation is inserted into a genome of the Photorhabdus temperata temperata through homologous recombination, thereby obtaining recombinant Photorhabdus temperata temperata, which are homologous recombinants. At this time, the homologous recombinants are formed at the overlapping sequences involved in replacing the promoters.

Bacteria showing the characteristic red color of the Photorhabdus temperata temperata are preferably spread on LB agar medium containing kanamycin and cultured in a temperature range of 28° C. to 32° C. for 1 to 3 days to obtain a single colony. It is more preferable for the bacteria to be cultured at a temperature of 30° C. for 2 days.

The recombinant Photorhabdus temperata temperata may be confirmed to have sequencing primers by a PCR analysis using mutation-specific primers.

When analyzed by PCR, wild Photorhabdus temperata temperata were found to produce a PCR product of 500 bp. However, recombinant Photorhabdus temperata temperata with sequencing primers were found to produce two PCR products: a 500-bp PCR product and a 5,823-bp long PCR product containing an insertion site.

As described above, the recombinant Photorhabdus temperata temperata obtained in an example of the present disclosure was deposited at the National Institute of Agricultural Sciences under accession number KACC 92578P on Nov. 29, 2023.

Expression of the gxpS gene in the recombinant Photorhabdus temperata temperata may be induced with arabinose. Expression of the gxpS gene is preferably induced by adding 0.2% arabinose to the Photorhabdus temperata temperata culture medium when an OD600 value reaches up to 0.6.

In the recombinant Photorhabdus temperata temperata, the expression level of the gxpS gene changes depending on the presence of the inducer L-arabinose. When the inducer L-arabinose is added, the expression level of the gxpS gene in the recombinant Photorhabdus temperata temperata is significantly higher than that in the wild Photorhabdus temperata temperata. The recombinant Photorhabdus temperata temperata, which show an increased expression level of the gxpS gene, produce large quantities of four types of GXPs: GXP-A, GXP-B, GXP-C, and GXP-D.

In another aspect of the present disclosure, a method of producing GXPs using the recombinant entomopathogenic bacteria is provided. The GXP production may be regulated by arabinose as described above.

In a further aspect of the present disclosure, a method of controlling pests is provided, the method including treating pests with the recombinant entomopathogenic bacteria or GXP-A.

The GXP-A is preferably produced by the same method as above while insecticidal functions of the GXP-A have already been confirmed.

GXPs obtained by the gxpS gene expression act to suppress immunity of pests. Thus, pests may be effectively controlled by using this immunosuppressive effect of the GXPs or recombinant Photorhabdus temperata temperata transformed to produce large quantities of the GXPs.

The pests subject to control by the GXP-A or recombinant entomopathogenic bacteria are not particularly limited, but preferably include Spodoptera exigua, Plutella xylostella, Frankliniella occidentalis, and Aedes albopictus.

In a still yet further preferred embodiment, the recombinant entomopathogenic bacteria or GXP-A may be co-treated with Bacillus thuringiensis. The recombinant entomopathogenic bacteria may increase the insecticidal power of Bacillus thuringiensis by suppressing insect immunity.

Known methods such as feeding, spraying, and contact may be applied for the treatment without limitation.

### EXAMPLES

The present disclosure will be described in detail below through examples. However, these examples are illustrative of the present disclosure, and the scope of the present disclosure is not limited thereto.

### Example 1

### Bacterial Culture

A Photorhabdus temperata temperata (Ptt) strain with accession number KACC91042 was used after having been obtained from the Korea RDA, Agricultural Resource Center (KACC, Jeon ju, Korea). The Photorhabdus temperata temperata strain was cultured using a TSB medium (Difco, Sparks, MD) in a shaking incubator at a temperature of 28° C. and a shaking speed of 180 rpm for 72 hours.

### Example 2

### Insect Farming

Spodoptera exigua were used as host insects. Spodoptera exigua were obtained from larvae, which had been collected from green onion fields in Andong, Gyeongsangbuk-do, and was reared in a temperature range of 27±1° C. using artificial feed (Goh et al., 1990). During the rearing process, Spodoptera exigua larvae evolved into adults through five stages of larval stages (L1 to L5). The adults were reared using sugar solution (10%).

### Example 3

### Synthesis of GXP Peptides

Four GXPs, GXP-A, GXP-B, GXP-C, and GXP-D, were synthesized by Anygen, Inc. (Gwangju, Korea). The GXP-A was synthesized to have a cyclic structure with the amino acid sequence of [D-Leu/L-Leu/D-Val/L-Leu/D-Phe]. The GXP-B was synthesized to have a cyclic structure with the amino acid sequence of [D-Leu/L-Leu/D-Leu/L-Leu/D-Phe]. The GXP-C was synthesized to have a ring-shaped structure with the amino acid sequence of [D-Leu/L-Leu/D-Val/L-Leu/D-Leu]. The GXP-D was synthesized to have a ring-shaped structure with the amino acid sequence of [D-Leu/L-Leu/D-Leu/L-Leu/D-Leu].

The four synthesized GXP peptides were confirmed to have a purity of over 97% based on quality control, which was determined through MALDI-TOF and LC-UV analysis by the manufacturer.

The four synthetized peptides were prepared to have a concentration of 1 mM using dimethyl sulfoxide (DMSO), and the prepared peptides were stored in a freezer, diluted with DMSO, and used for analysis.

### Example 4

RNA Extraction and cDNA Preparation With Photorhabdus Temperata Temperata

The Photorhabdus temperata temperata cultured in Example 1 were centrifuged, and a supernatant was removed. Thereafter, 1 mL of Trizol reagent (Invitrogen, Carlsbad, CA, USA) was added, and RNAs were extracted according to the procedure described in the manufacturer's instructions.

Each extracted RNA was resuspended in 50 μL of DEPC solution, and the concentration of each of the extracted RNAs was measured using a spectrophotometer (Nanodrop, Thermo Fisher Scientific).

CDNAs were synthesized using RT premix (Intron Biotechnology, Seoul, Korea) containing 400 ng of RNA and random primers.

A RT-PCR reaction amplified cDNAs from a specific target gene using gene-specific primers and Taq polymerase (GeneALL, Seoul, Korea). The RT-PCR reaction was performed with an initial denaturation at a temperature of 95° C. for 5 minutes followed by 35 amplification cycles of 95° C. for 1 minute, 53° C. to 55° C. for 1 minute, and 72° C. for 1 minute. PCR products were analyzed by 1% agarose gel electrophoresis to confirm the presence of amplified products.

An RT-qPCR reaction was performed using Power Syber Green PCR Master Mix (Toyobo, Osaka, Japan) with gene-specific primers according to the instructions of Bustin et al. (2009). A quantitative analysis was performed using a comparative CT method (Livak and Schmittgen, 2001).

### Example 5

### Extraction of Photorhabdus Temperata Temperata Secondary Metabolites and Confirmation of Relative Expression Levels of GxpS Gene

Secondary metabolites of the Photorhabdus temperata temperata cultured in Example 1 were extracted. First, the Photorhabdus temperata temperata were cultured for 72 hours at a temperature of 28° C. using 1 L of TSB medium at a shaking speed of 180 rpm. An obtained Photorhabdus temperata temperata culture medium was centrifuged at 12,500×g for 20 minutes at a temperature of 4° C., and 1 L of ethyl acetate was mixed with a supernatant. An organic phase was collected using a separatory funnel. This process was repeated three times. 3 L of the ethyl acetate layer was concentrated at a temperature of 30° C. using a rotary evaporator (N-1110 Eyela, Tokyo, Japan). The resulting dry pellet containing the metabolites was weighed, resuspended in DMSO, and diluted to have a final concentration of 100 ppm.

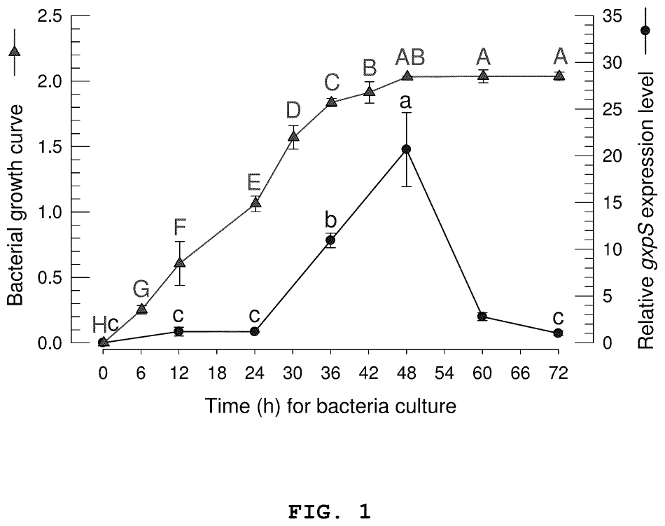

Photorhabdus temperata temperata growth (measured by OD600) and relative expression levels of the gxpS gene depending on the culture time over the culture periods in the TSB medium are shown in FIG. 1.

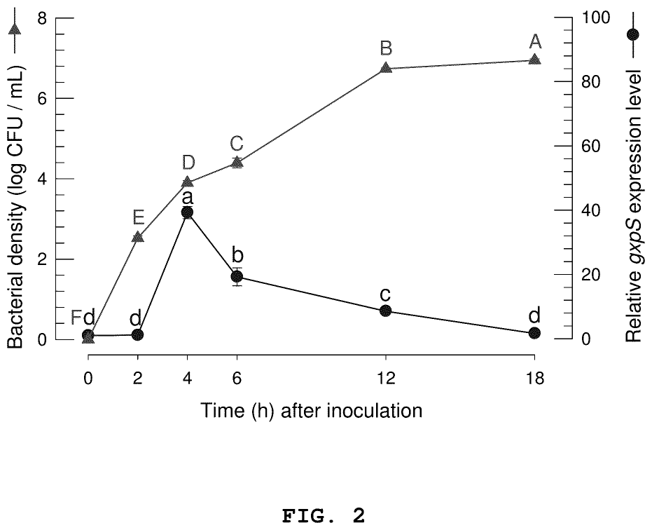

In addition, the results of confirming the Photorhabdus temperata temperata growth (measured in CFU) and the relative expression levels of the gxpS gene in a hemocoel of the Spodoptera exigua are shown in FIG. 2.

As shown in the results in FIGS. 1 and 2, the gxpS gene was confirmed to be expressed at various expression levels during various culture periods while the Photorhabdus temperata temperata were growing in both the TSB medium and the hemocoel of the Spodoptera exigua.

When cultured in the TSB medium, the Photorhabdus temperata temperata showed exponential growth after a lag phase for the first 6 hours and reached a stationary phase after 24 hours. The gxpS gene showed a basic expression level in the logarithmic growth phase of the Photorhabdus temperata temperata, but its expression increased rapidly in the stationary phase.

When cultured in the hemocoel of the Spodoptera exigua, the Photorhabdus temperata temperata growth was similar to that in the TSB medium, but a growth rate was higher in the case of the hemocoel of the Spodoptera exigua. The gxpS gene was highly expressed in the early logarithmic growth stage of Spodoptera exigua.

### Example 6

### Evaluation of GXP Production in Photorhabdus Temperata Temperata Culture Media

GXP production in Photorhabdus temperata temperata culture media was evaluated using four different GXP peptide standards (GXP-A to GXP-D) synthesized in Example 3.

Extracts of the Photorhabdus temperata temperata culture media were used as samples. The samples were analyzed by liquid chromatography-tandem mass spectrometry (LC-MS/MS).

The samples were filtered through a disposable membrane filter device (PVDF, 0.2 μm pore size, Thermo Fisher Scientific). Thereafter, the samples were diluted by being mixed with methanol at a ratio of 1:1 before the LC-MS analysis. To obtain a calibration curve, a six-point calibration solution made from a mixture of four GXPs (GXP-A to GXP-D) at a concentration of 0 to 100 μm was prepared using DMSO.

The LC-MS/MS analysis was performed using a 5600+ TripleTOF electrospray ionization triple quadrupole-time of flight mass spectrometer (AB Sciex, Framingham, MA, USA) coupled to a Prominence 20AD series HPLC (Shimadzu, Japan).

For GXP measurement, 2 μL of the samples were injected into KINETEX F5 columns (2.6 μm, 100′3 mm i.d., Phenomenex, Torrance, CA, USA) and analyzed. Ion transitions (m/z) were identified as follows: GXP-A (586.4>473.3), GXP-B (600.4>487.3), GXP-C (552.4>439.3), GXP-D (566.4>453.2), and GXP-A* (593.30>480.3). The source voltage was 5.5 kV and the temperature was 500° C. Other ionization and fragmentation parameters were optimized by monitoring MS signals prior to the sample analysis.

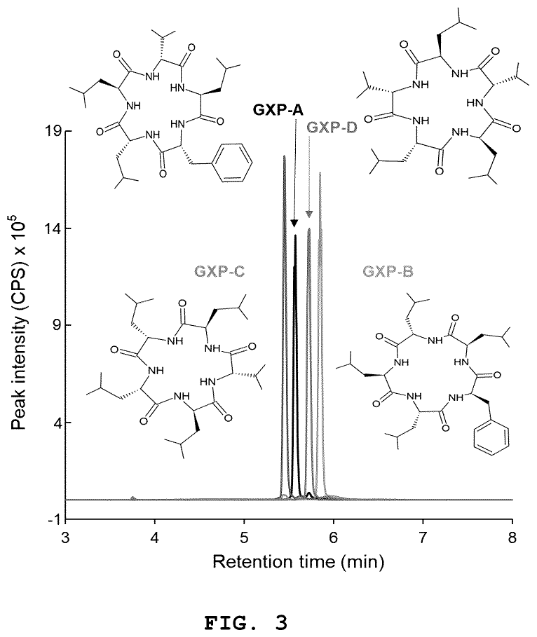

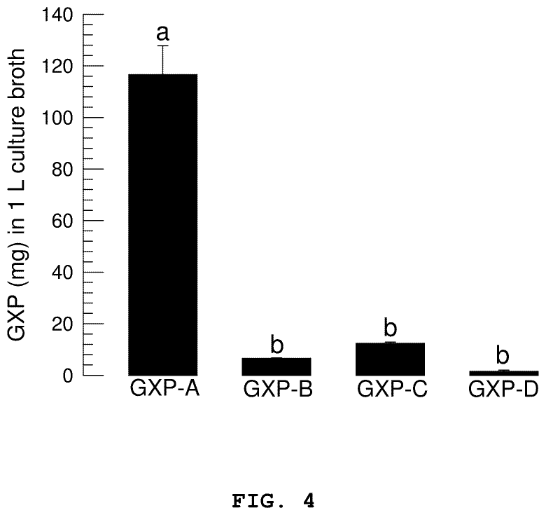

The chromatograms of the four GXPs (GXP-A, GXP-B, GXP-C, and GXP-D), identified on the basis of their chemical structures using LC-MS/MS and MS/MS spectra, are shown in FIG. 3. A graph showing quantification of the four GXPs contained in the Photorhabdus temperata temperata culture media, which was cultured for 72 hours in the TSB medium, is shown in FIG. 4.

The four GXPs were detected in the extracts of the Photorhabdus temperata temperata culture media under the same LC-MS/MS analysis conditions. Among the four GXPs, GXP-A was contained the most in the extracts of the Photorhabdus temperata temperata culture media, and a concentration of the GXP-A was about 135 μg/L. This concentration is higher by 7 times or more than those of the other three GXPs.

### Example 7

### Preparation of Recombinant Photorhabdus Temperata Temperata

To determine roles of gxpS gene expression in GXP production in the Photorhabdus temperata temperata, the promoter of the gxpS gene in the Photorhabdus temperata temperata was replaced with an arabinose-inducible araBAD promoter, thereby recombinant Photorhabdus temperata temperata showing different expression levels of the gxpS gene were prepared.

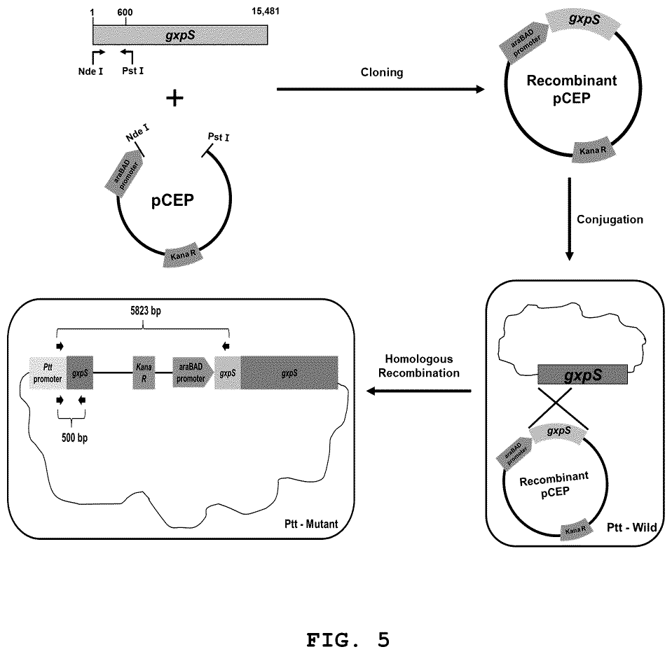

FIG. 5 shows a diagram showing a process of producing the recombinant Photorhabdus temperata temperata.

First, an initial ORF region (600 bp) located at the 5′ side of the gxpS gene in the Photorhabdus temperata temperata was amplified by using a forward primer with a sequence (CATATGATGAAAGACAGTATTACCAG) containing Nde I restriction enzyme (underlined) and a reverse primer with a sequence (CTGCAGCATGATATACGCCGGCCCGG) containing PstI restriction enzyme.

The amplified initial ORF regions (600 bp) of the gxpS gene, which were generated PCR products, were cloned into pCEP plasmid vectors (Bode et al., 2015) under the arabinose inducible araBAD promoter. The obtained recombinant pCEP vectors were inserted into E. coli S17 competent cells and transformed.

The Photorhabdus temperata temperata (60 μL) and transformed E. coli S17 culture media (20 μL) were co-cultured at a temperature of 30° C. in LB (Luria-Bertani) medium until an optical density of each of the mixtures reached 0.6 to 0.7 at a wavelength of 600 nm, thereby the two bacteria were conjugated. Through the bacterial conjugation between the Photorhabdus temperata temperata and E. coli, the recombinant pCEP vectors were transferred to wild Photorhabdus temperata temperata. Through homologous recombination, the recombinant pCEP vectors were inserted into a genome of the Photorhabdus temperata temperata. As a result, recombinant Photorhabdus temperata temperata, which were homologous recombinants, were formed at overlapping sequences involved in replacing promoters.

Bacteria showing the characteristic red color of the Photorhabdus temperata temperata were spread on LB agar medium containing kanamycin and were cultured at a temperature of 30° C. for 2 days, thereby obtaining a single colony.

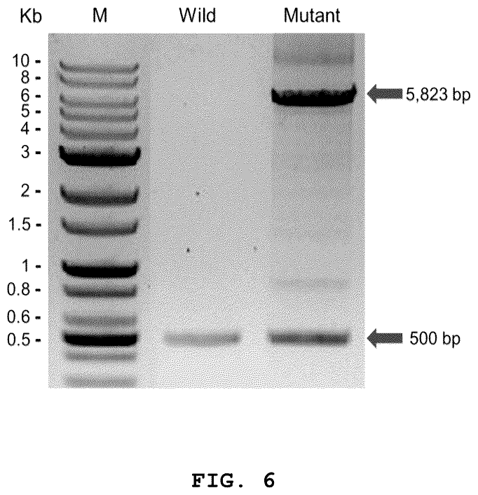

The single colony obtained through the process was analyzed by PCR using Photorhabdus temperata temperata mutation-specific primers, and the results are shown in FIG. 6.

FIG. 6 shows results of confirming the PCR products of mutants with sequencing primers. In the recombinant Photorhabdus temperata temperata (Ptt-Mutant), arrows indicate sequencing primers.

As shown in FIG. 6, the wild Photorhabdus temperata temperata produced a PCR product of 500 bp. However, the recombinant Photorhabdus temperata temperata with sequencing primers produced two PCR products: a 500-bp PCR product and a 5,823-bp long PCR product containing an insertion site.

### Example 8

### Confirmation of Expression Levels of GxpS Gene in Recombinant Photorhabdus Temperata Temperata

Expression levels of the gxpS gene in wild Photorhabdus temperata temperata and recombinant Photorhabdus temperata temperata were confirmed as follows.

To induce gxpS gene expression in the recombinant Photorhabdus temperata temperata, in a recombinant Photorhabdus temperata temperata experimental group, 0.2% L-arabinose (L-arabinose, ARA) was added to the culture medium when an OD600 value for the Photorhabdus temperata temperata culture medium reached up to 0.6. Arabinose was not added to a recombinant Photorhabdus temperata temperata comparison group.

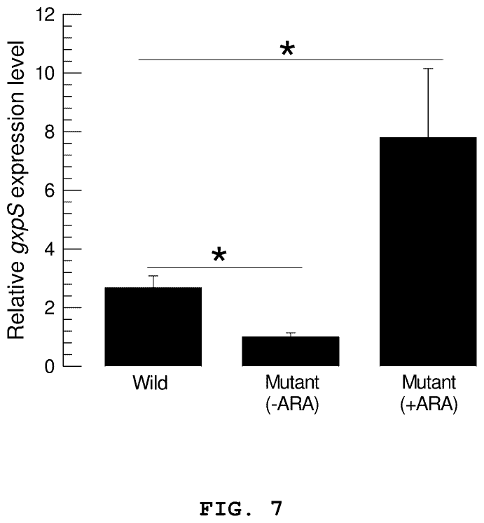

After culturing, the relative expression levels of the gxpS gene in the wild Photorhabdus temperata temperata and recombinant Photorhabdus temperata temperata were confirmed. The results are shown in FIG. 7. In FIG. 7, “−ARA” indicates the recombinant Photorhabdus temperata temperata comparison group without arabinose added, meanwhile “+ARA” indicates the recombinant Photorhabdus temperata temperata experimental group with arabinose added.

As shown in the results of FIG. 7, the expression level of the gxpS gene in the recombinant Photorhabdus temperata temperata changed depending on the presence of L-arabinose, an inducer. In the absence of the inducer L-arabinose, the expression level of the gxpS gene in the recombinant Photorhabdus temperata temperata was significantly lower than that in the wild Photorhabdus temperata temperata (F=15.55; df=1,4; P=0.0169). However, when the inducer L-arabinose was added, the recombinant Photorhabdus temperata temperata upregulated the expression of the gxpS gene to a much greater extent than wild Photorhabdus temperata temperata (F=24.49; df=1,4; P=0.0078).

### Example 9

### Confirmation of GXP Production in Recombinant Photorhabdus Temperata Temperata

GXP production in wild Photorhabdus temperata temperata and recombinant Photorhabdus temperata temperata was confirmed as follows.

The wild Photorhabdus temperata temperata and recombinant Photorhabdus temperata temperata were cultured at a temperature of 28° C. for 72 hours using a TSB medium. In a recombinant Photorhabdus temperata temperata experimental group, 0.2% L-arabinose (ARA) was added to the culture medium when an OD600 value for the Photorhabdus temperata temperata culture medium reached up to 0.6. Arabinose was not added to a recombinant Photorhabdus temperata temperata comparison group.

After culturing, the GXP production was confirmed in the wild Photorhabdus temperata temperata and recombinant Photorhabdus temperata temperata.

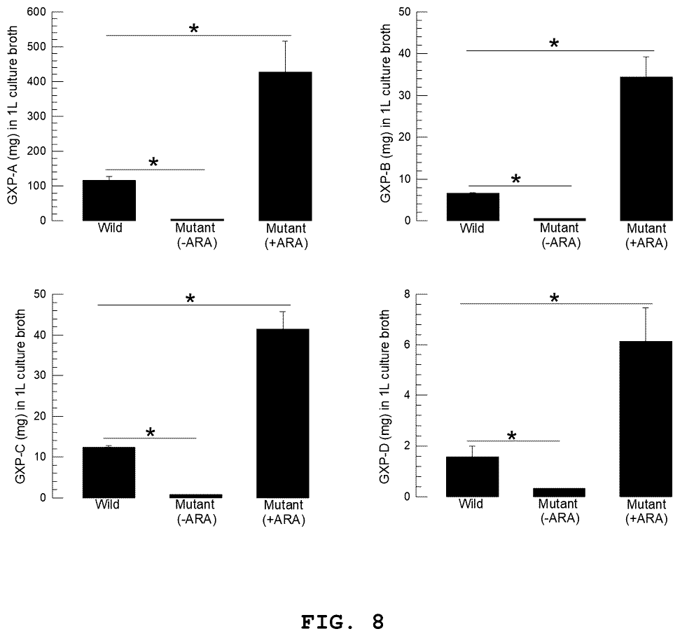

The amounts of the four GXPs produced in the culture media of wild Photorhabdus temperata temperata, recombinant Photorhabdus temperata temperata without arabinose added, and recombinant Photorhabdus temperata temperata with arabinose added were compared, and the results are shown in FIG. 8. In FIG. 8, “−ARA” indicates the recombinant Photorhabdus temperata temperata comparison group without arabinose added, meanwhile “+ARA” indicates the recombinant Photorhabdus temperata temperata experimental group with arabinose added. Each process was repeated three times. The asterisks indicate a significant difference between the two means at Type I error=0.05 (LSD test).

As shown in the results of FIG. 8, the GXP production in the recombinant Photorhabdus temperata temperata changed depending on the presence of L-arabinose, an inducer. In the absence of the inducer L-arabinose, the expression level of the gxpS gene in the recombinant Photorhabdus temperata temperata produced significantly lower amounts of GXP-A than the wild Photorhabdus temperata temperata (F=97.49; df=1,4; P=0.0006). However, when the inducer L-arabinose was added, the recombinant Photorhabdus temperata temperata produced a much higher amount of GXP-A than the wild Photorhabdus temperata temperata (F=12.13; df=1,4; P=0.0253). Similar changes were confirmed in three other GXPs of the recombinant Photorhabdus temperata temperata.

### Example 10

### Hemocyte-Spreading Analysis

To confirm a relationship between an expression regulation of the gxpS gene and an immunosuppressive activity of the Photorhabdus temperata temperata, a hemocyte adhesion reaction was analyzed as follows through a hemocyte-spreading assay using Spodoptera exigua larvae, which responded to infection with Photorhabdus temperata temperata.

“Wild” indicates wild Photorhabdus temperata temperata with an original gxpS promoter, and “Mutant” indicates recombinant Photorhabdus temperata temperata with the gxpS promoter replaced by the araBAD promoter. To induce the gxpS gene expression in the recombinant Photorhabdus temperata temperata, 0.2% L-arabinose (ARA) was added to the culture medium when an OD600 value for the Photorhabdus temperata temperata culture medium reached up to 0.6. “−ARA” indicates a recombinant Photorhabdus temperata temperata comparison group without arabinose added, meanwhile “+ARA” indicates a recombinant Photorhabdus temperata temperata experimental group with arabinose added. In all experiments, organic extracts from the Photorhabdus temperata temperata culture media were used. “Con” indicates a solvent (DMSO) treatment group.

The Spodoptera exigua larvae used were 3-day-old L5 larvae. 1 μL of cultured E. coli (5×10⁷ cells/mL) and 1 μL of each of the Photorhabdus temperata temperata extracts (100 ppm) were injected into the bloodstream of the Spodoptera exigua larvae through the prolegs of the Spodoptera exigua larvae using a microsyringe (Hamilton, Reno, NV, USA). The injected Spodoptera exigua larvae were incubated at a temperature of 25° C. for 8 hours. Thereafter, larval blood cells were observed using a 200× fluorescence microscope to confirm F-actin growth in response to each of the Photorhabdus temperata temperata extracts. Five larvae were used in each experiment, and each experiment was repeated three times.

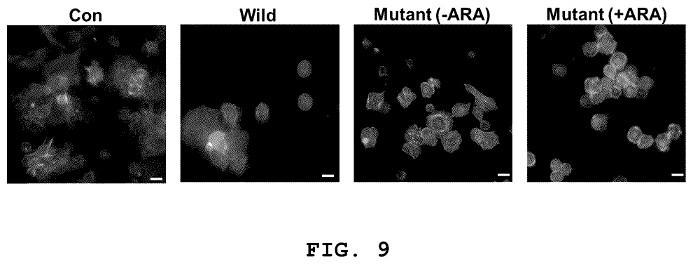

The results of the hemocyte-spreading analysis performed by observing F-actin growth in response to each of the Photorhabdus temperata temperata extracts are shown in FIG. 9. F-actin filaments were specifically recognized by FITC-tagged phalloidin (green), and nuclei were stained with DAPI (blue). A scale bar represented 10 mm.

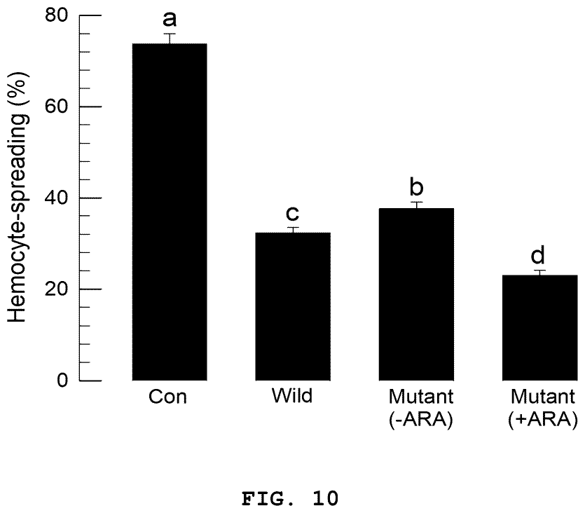

About 250 μL of hemolymph was obtained by cutting the abdominal legs of the Spodoptera exigua L5 larvae, and the 250 μL of hemolymph was mixed with 350 μL of cold anticoagulant buffer solution. Afterward, the mixture was centrifuged at 1,000 rpm for 2 minutes to remove 400 μL of a supernatant. The remaining blood cells were resuspended in TC-100 insect culture medium (Hyclone, Daegu, Korea). A total of 10 μL of reaction mixture each prepared with 9 μL of blood cell suspension and 1 μL of each of the Photorhabdus temperata temperata extracts (100 ppm) was placed on a glass slide. After culturing for 30 minutes in dark room conditions at room temperature, blood cells were observed at 400× magnification using a phase contrast microscope (DM2500, Leica, Wetzlar, Germany). Diffuse hemocytes featured cytoplasmic expansion beyond cell boundaries. Each experiment was repeated three times. The results of this hemocyte-spreading analysis are shown in FIG. 10.

As shown in the results of FIGS. 9 and 10, all Photorhabdus temperata temperata extracts significantly inhibited cellular immune responses measured by the hemocyte adhesion reaction. However, there was variation among the Photorhabdus temperata temperata extracts. The recombinant Photorhabdus temperata temperata extract (−ARA) without the inducer added had a less immune response suppression effect than the wild Photorhabdus temperata temperata extract. Meanwhile, the recombinant Photorhabdus temperata temperata extract (+ARA) with the inducer added had a higher immune response suppression effect than the wild Photorhabdus temperata temperata extract.

### Example 11

### Nodulation Assay and Phenoloxidase (PO) Activity Analysis

To confirm a relationship between an expression regulation of gxpS gene and an immunosuppressive activity of the Photorhabdus temperata temperata, vesicle formation responses and phenoloxidase activity in response to infection with the Photorhabdus temperata temperata were analyzed as follows using Spodoptera exigua larvae.

“Wild” indicates wild Photorhabdus temperata temperata with an original gxpS promoter, and “Mutant” indicates recombinant Photorhabdus temperata temperata with the gxpS promoter replaced by the araBAD promoter. To induce the gxpS gene expression in the recombinant Photorhabdus temperata temperata, 0.2% L-arabinose (ARA) was added to the culture medium when an OD600 value for the Photorhabdus temperata temperata culture medium reached up to 0.6. “−ARA” indicates a recombinant Photorhabdus temperata temperata comparison group without arabinose added, meanwhile “+ARA” indicates a recombinant Photorhabdus temperata temperata experimental group with arabinose added. In all experiments, organic extracts from the Photorhabdus temperata temperata culture media were used. “Con” indicates a solvent (DMSO) treatment group.

As host insects, Spodoptera exigua larvae were used, and 3-day-old L5 larvae were used as the Spodoptera exigua larvae. 1 μL of cultured E. coli (5×10⁷ cells/mL) and 1 μL of each of the extracts of Photorhabdus temperata temperata culture media were injected into the bloodstream of the Spodoptera exigua larvae through the prolegs of the Spodoptera exigua larvae using a microsyringe (Hamilton, Reno, NV, USA).

The injected Spodoptera exigua larvae were incubated at a temperature of 25° C. for 8 hours. Afterward, the Spodoptera exigua larvae were dissected, and melanized vesicles obtained from the Spodoptera exigua larvae were counted at 50× magnification using a microscope (Stemi SV11, Zeiss, Jena, Germany). Five larvae were used in each experiment, and each experiment was repeated three times.

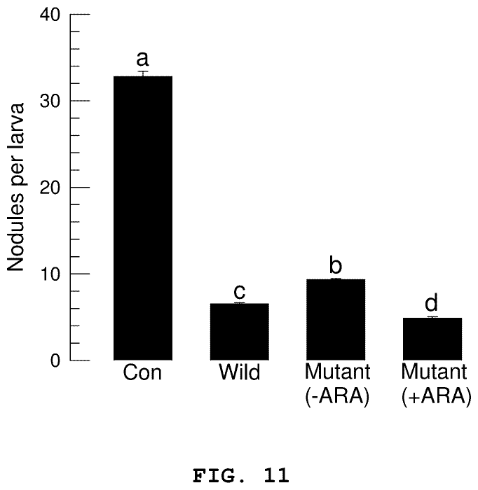

The results of analyzing the vesicle formation responses shown in response to the infection with the Photorhabdus temperata temperata are shown in FIG. 11.

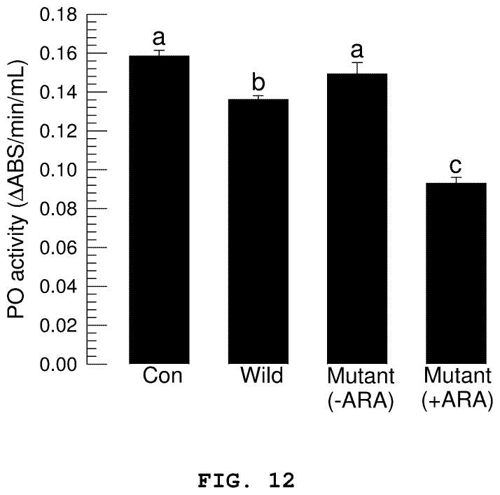

In addition, the results of analyzing the activity of phenoloxidase shown in response to the infection with the Photorhabdus temperata temperata are shown in FIG. 12.

(If possible, it is recommended to provide a more detailed explanation of the experimental process for confirming the activity of phenoloxidase.)

Phenomenons similar to those shown in Example 10 were observed in the evaluation of the immune responses measured as a result of confirming the vesicle formation reactions in FIG. 11 and the phenoloxidase (PO) activity in FIG. 12.

### Example 12

### Comparison of Insecticidal Activity of Wild and Recombinant Photorhabdus Temperata Temperata

Insecticidal activity of wild and recombinant Photorhabdus temperata temperata against Spodoptera exigua was compared as follows.

The Spodoptera exigua larvae used were 3-day-old L4 larvae. 1 μL of each of the culture media containing 1.5×10² CFU of Photorhabdus temperata temperata per larva was injected into the bloodstream of the Spodoptera exigua larvae through the prolegs of the Spodoptera exigua larvae using a microsyringe (Hamilton, Reno, NV, USA). After the larvae were treated with each of the culture media, culture was performed for 3 days.

“Wild” indicates wild Photorhabdus temperata temperata with an original gxpS promoter, and “Mutant” indicates recombinant Photorhabdus temperata temperata with the gxpS promoter replaced by the araBAD promoter. “Mutant+GXP-A” indicates that GXP-A (10 μg/larva) was injected with recombinant Photorhabdus temperata temperata, which has the gxpS promoter replaced by the araBAD promoter. In all experiments, organic extracts from the Photorhabdus temperata temperata culture media were used. Ten larvae were used in each experiment, and each experiment was repeated three times.

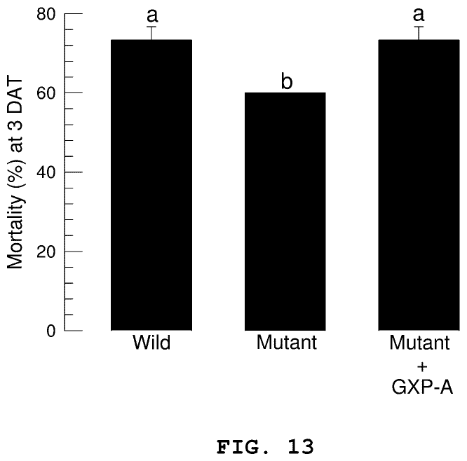

The results of confirming the insecticidal activity of the wild and recombinant Photorhabdus temperata temperata are shown in FIG. 13.

As shown in FIG. 13, all Photorhabdus temperata temperata showed significant insecticidal activity within 3 days of treatment. However, the recombinant Photorhabdus temperata temperata showed significantly lower insecticidal activity than the wild Photorhabdus temperata temperata. When the recombinant Photorhabdus temperata temperata was co-injected with GXP-A, the insecticidal activity was significantly increased.

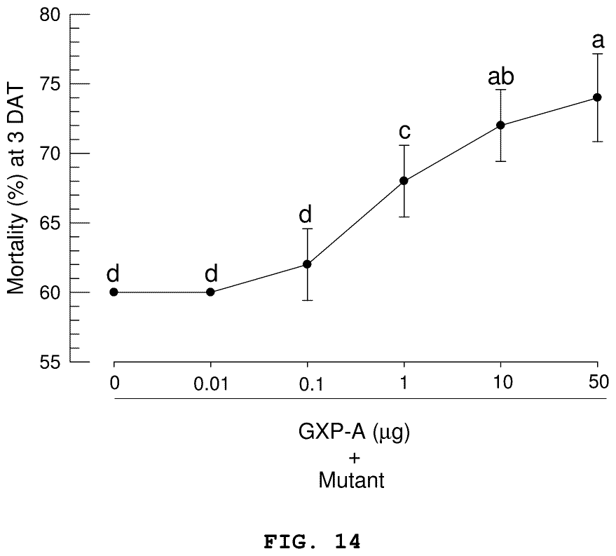

0.01 μg, 0.1 μg, 1 μg, 10 μg, and 60 μg of GXP-A per larva were added to the recombinant Photorhabdus temperata temperata (1.5×10² CFU/larva) culture medium, and each of the GXP-A/recombinant Photorhabdus temperata temperata mixtures were administered to the larvae. Dose-mortality rates depending on the different doses of GXP-A, each of which was added to the recombinant Photorhabdus temperata temperata (1.5×10² CFU/larva) culture medium, were confirmed, and the results are shown in FIG. 14. In the dose-mortality curve of FIG. 14, different letters above the standard deviation bar indicate significant differences between means at Type I error=0.05 (LSD test).

As shown in the results of FIG. 14, the insecticidal activity of the recombinant Photorhabdus temperata temperata increased in a dose-dependent manner with the addition of GXP-A.

The recombinant entomopathogenic bacteria using the promoter replacement techniques of the present disclosure described above and the preparation method and use thereof are exemplary. Those of ordinary skill in the technical field to which the present disclosure pertains will appreciate that various modifications and other equivalent embodiments are possible. Therefore, it will be well understood that the present disclosure is not limited to the forms mentioned in the description of the present disclosure above. The true scope of technical protection of the present disclosure should be determined by the technical spirit of the claims. It should be understood to include all modifications, equivalents, and substitutes within the spirit and scope of the present disclosure as defined by the claims of the present disclosure. Additionally, terms or words used in this specification and claims should not be construed as limited to their usual or dictionary meanings. On the basis of the principle that the inventor(s) can appropriately define the concept of the terms to explain his or her invention in the best way, the terms or words should be interpreted with meaning and concept consistent with the technical idea of the present disclosure.

### Accession Number

Name of Depository Institution: Microbial Bank, National Institute of Agricultural Sciences, Rural Development Administration (RDA), Korean Agricultural Culture Collection (KACC)

Accession Number: KACC92578P

Date of Deposit: 2023 Nov. 29

### Sequence List

Sequence list electronic file attached

What is claimed is:

1. Recombinant entomopathogenic bacteria, wherein a promoter of a GXP synthase gene (gxpS gene) is replaced in entomopathogenic bacteria with an arabinose inducible promoter using a promoter replacement technique.

2. The recombinant entomopathogenic bacteria of claim 1, wherein the entomopathogenic bacteria are Photorhabdus temperata temperata.

3. The recombinant entomopathogenic bacteria of claim 2, wherein the recombinant entomopathogenic bacteria are Photorhabdus temperata temperata deposited as KACC 92578P.

4. The recombinant entomopathogenic bacteria of claim 1, wherein the arabinose inducible promoter is araBAD.

5. The recombinant entomopathogenic bacteria of claim 1, wherein the recombinant entomopathogenic bacteria mass-produce GXPs under control of the arabinose.

6. A method of preparing recombinant entomopathogenic bacteria, the method comprising:
amplifying an initial ORF region of a GXP synthase gene (gxpS gene) of entomopathogenic bacteria;
obtaining a recombinant vector by cloning the amplified initial ORF region into a vector under an arabinose inducible promoter;
obtaining a transformed E. coli by bringing the recombinant vector to be inserted into E. coli;
transferring the recombinant vector to the entomopathogenic bacteria by co-culturing the entomopathogenic bacteria and the transformed E. coli and facilitating bacterial conjugation thereof; and
obtaining recombinant entomopathogenic bacteria, which are homologous recombinants, by bringing the recombinant vector to be inserted into a genome of the entomopathogenic bacteria through homologous recombination.

7. The method of claim 6, further comprising obtaining a single colony by culturing the recombinant entomopathogenic bacteria.

8. The method of claim 6, wherein the arabinose inducible promoter is araBAD.

9. The method of claim 6, wherein homologous recombinants are formed at overlapping sequences involved in replacing promoters by bringing the recombinant vector to be inserted into a genome of the entomopathogenic bacteria through homologous recombination.

10. The method of claim 6, wherein the recombinant entomopathogenic bacteria are Photorhabdus temperata temperata.

11. The method of claim 10, wherein the recombinant entomopathogenic bacteria are Photorhabdus temperata temperata deposited as KACC 92578P.

12. The method of claim 10, wherein the single colony is obtained by spreading the recombinant Photorhabdus temperata temperata on LB agar medium containing kanamycin and culturing the recombinant Photorhabdus temperata temperata in a temperature range of 28° C. to 32° C. for 1 to 3 days.

13. A method of producing GXPs using the recombinant entomopathogenic bacteria of claim 1.

14. The GXP production method of claim 13, wherein the GXP production is controlled by arabinose.

15. A method of controlling pests, the method comprising treating pests with the recombinant entomopathogenic bacteria of claim 1 or GXP-A.

16. The pest control method of claim 15, wherein the pests comprise any one selected from the group consisting of Spodoptera exigua, Plutella xylostella, Frankliniella occidentalis, and Aedes albopictus.


SEQUENCE LISTING (2 sequences)

SEQ ID NO: 1 - DNA, 26 bp, synthetic construct
  misc_feature 1..26: note = Forward primer
  source 1..26: mol_type = other DNA
catatgatgaaagacagtattaccag

SEQ ID NO: 2 - DNA, 26 bp, synthetic construct
  misc_feature 1..26: note = Reverse primer
  source 1..26: mol_type = other DNA
ctgcagcatgatatacgccggcccgg